(19) United States
(12) Patent Application Publication
(10) Pub. No.: US 2019/0374958 A1
(43) Pub. Date: Dec. 12, 2019

(54) AIRTIGHT APPARATUS FOR SEPARATING AND PURIFYING EXTRACT

(71) Applicant: APINTECH, INC., Seongdong-gu, Seoul (KR)

(72) Inventor: Gi Young JANG, Gangnam-gu, Seoul (KR)

(21) Appl. No.: 16/488,410

(22) PCT Filed: May 8, 2017
(86) PCT No.: PCT/KR2017/004750

§ 371 Date: Aug. 23, 2019

(30) Foreign Application Priority Data
Feb. 27, 2017 (KR) 10-2017-0025346

(51) Int. Cl. B03C 1/28; B03C 1/12; C07K 1/14

(52) U.S. Cl. CPC B03C 1/28; B03C 2201/18; C07K 1/14; B03C 1/12

(57) ABSTRACT

The present invention relates to an airtight apparatus for separating and purifying an extract, comprising: a chamber cartridge having a plurality of chambers for receiving samples and magnetic beads for separation of biological materials; a cover having a bore capable of being introduced into any one of the plurality of chambers, covering the plurality of chambers, and being liftably coupled to the chamber cartridge; a sealing member provided between the cover and the chamber cartridge to maintain airtightness between the cover and a housing; a cover driving part for raising and lowering the cover such that the bore ascends and descends in the chamber; a rod for inserting into and withdrawing from the bore to attach/detach the biological materials adsorbed on the magnetic beads received in the chamber to/from the surface of the bore or to introduce/discharge the samples with the biological materials separated and the magnetic beads into/from the bore; a rod driver for inserting and withdrawing the rod into/from the bore; and a bore moving part for moving the bore to one of the plurality of chambers.

### TECHNICAL FIELD

The present invention relates to an airtight apparatus for separation and purification of an extract and, more particularly, an airtight apparatus for separation and purification of an extract, capable of separating or purifying a variety of biological materials such as nucleic acid or antibodies, etc.

### BACKGROUND

Nucleic acid extracted from various biological samples such as blood or plasma is an important starting material in many areas such as medical diagnostics, molecular biology and pathological medicine.

In recent years, due to spread of real time PCR that is able to detect nucleic acid amplification during PCR using a fluorescent material as well as a polymerase chain reaction (PCR) enabling nucleic acid amplification, extraction of nucleic acid is frequently required. Further, nucleic acid extraction equipment with implementing an automated extraction process is widely used.

Methods applied to such automated nucleic acid extraction equipment may be generally classified into two kinds of processes, for example: a process of introducing a lysed sample to a tube into which a silica filter is inserted, conducting centrifugation to adhere nucleic acid to the silica filter and purifying the same; and another process of introducing magnetic beads possibly adhered to nucleic acid in a tube wherein a lysed sample is included, and then, purifying the same (U.S. Pat. No. 4,787,971 (1988), U.S. Pat. No. 6,103,195 (2000), U.S. Pat. No. 4,871,675 (1989). U.S. Pat. No. 8,062,533 (2011), U.S. Pat. No. 4,270,921 (1981). U.S. Pat. No. 5,200,084 (1993), PCT international Patent Laid-Open Publication No. WO 2001/68263 A1 (2001)).

However, both of these processes involve exposure to air during extraction and thus are not free from drawbacks including, in particular, infection of a subject under experiment by an infectious agent such as bacteria, viruses, etc. in a sample, cross-contamination between samples, and possibility of nucleic acid contamination in air.

### BRIEF DESCRIPTION OF THE DRAWINGS

FIG. 1 is a configuration diagram illustrating the airtight apparatus for separation and purification of an extract according to one embodiment of the present invention.

FIG. 2 is an exploded perspective view illustrating the chamber cartridge and the lid shown in FIG. 1.

FIG. 3 is a sectional perspective view illustrating an essential portion of the chamber cartridge and the lid shown in FIG. 1, in a combined state.

FIGS. 4 to 9 illustrate a process of separating and purifying biological materials using the airtight apparatus for separation and purification of an extract shown in FIG. 1.

FIG. 10 is a configuration diagram illustrating the airtight apparatus for separation and purification of an extract according another embodiment of the present invention.

FIG. 11 is an exploded perspective view illustrating the chamber cartridge, the lid and the rod shown in FIG. 10.

FIG. 12 is a sectional perspective view illustrating an essential portion of the chamber cartridge, the lid and the rod shown in FIG. 10, in a combined state.

FIGS. 13 to 18 illustrate a process of separating and purifying biological materials using the airtight apparatus for separation and purification of an extract shown in FIG. 10.

### DETAILED DISCLOSURE

### Technical Problem

The present invention is intended to solve the above problems, and an object of the present invention is to provide an airtight apparatus for separation and purification of an extract, which may perform separation and purification of an extract in an airtight state without exposing a sample to air, and may prevent infection of a subject under experiment by an infectious agent contained in the sample, introduction of foreign matter such as bacteria, viruses or nucleic acid possibly present in air, and/or cross-contamination of plural samples during extraction.

### Technical Solution

An object of the present invention is to provide an airtight apparatus for separation and purification of an extract, including: a chamber cartridge having a plurality of chambers for receiving a sample and magnetic beads for separation of a biological material; a lid having a bore through which the sample can be introduced into any one of the plurality of chambers, covering the plurality of chambers, and being lift-possibly coupled to the chamber cartridge; a sealing member provided between the lid and the chamber cartridge to keep a space between the lid and a housing airtight; a lid driver for lifting the lid up and down such that the bore ascends and descends in the chamber; a rod inserted into and withdrawn from the bore to attach/detach the biological material adsorbed onto the magnetic beads present in the chamber to/from the surface of the bore or to introduce/discharge the sample free of the biological material and the magnetic beds into/from the bores; a rod driver for inserting/withdrawing the rod into/from the bore; and a bore moving member for moving the bore to any one of the plurality of chambers.

In this case, a magnet for attaching/detaching the magnetic beads received in the bore to/from the surface of the bore may be further included.

The lid driver may include: a lid bracket to support the lid: a screw shaft for the lid (“lid screw shaft”) screw-rotatably coupled to the lid bracket to lift the same up and down; and a lid drive motor to rotate the lid screw shaft.

The rod driver may include, a first rod bracket for supporting the rod; a second rod bracket disposed apart from the first rod bracket at a distance; a bracket drive motor supported by the second rod bracket, having a drive shaft coupled to the first rod bracket, and rotating the first rod bracket; a screw shaft for the rod (“rod screw shaft”) screw-rotatably coupled to the second rod bracket to lift the same up and down; and a rod drive motor for rotating the rod screw shaft.

The bore moving member may include—a key protruding from the first rod bracket toward the lid; and a key coupling member disposed on the lid by which the key is engaged and released

A reciprocation member to reciprocate the lid driver and the rod driver in a transverse direction to the lifting direction of the bore may be further included. The reciprocation member may include: a main support bracket to support the lid driver and the rod driver: a main screw shaft screw-rotatably coupled to the main support bracket to reciprocate the main support bracket in a transverse direction to the lifting direction of the bore: and a main drive motor for rotating the main screw shaft.

An orbital shaker, on which the chamber cartridge is seated, to enable horizontal trajectory movement of the chamber cartridge, may be further included.

The orbital shaker may include: a shaking platform on which the chamber cartridge is disposed: and a shaking motor to conduct the horizontal trajectory movement of the shaking platform.

An air-vent part provided in the lid or the chamber cartridge to discharge the air between the lid and the chamber cartridge to the outside and/or introduce fresh air from the outside may be further included.

The air-vent part may further include a filter member to block introduction of foreign matter such as bacteria, viruses, nucleic acid, etc. and/or outflow of an aerosol including nucleic acid or relevant samples present therein.

The bore may have a tube shape opened at one side thereof.

The bore may have a cylindrical syringe shape opened at opposite sides.

A magnet provided below the rod to attach/detach the magnetic beads to/from the surface of the bore may be further included.

To a lower portion of the rod, an O-ring or a rubber ring may be adhered so that the rod may ascend and descend while keeping the inside of the bore airtight, thereby introducing/discharging the sample or the magnetic beads to/from the bores.

### Advantageous Effects

According to the present invention, it is possible to perform separation and purification of an extract in an airtight state without exposure of a sample to air, and to prevent infection of a subject under experiment by an infectious agent contained in the sample, introduction of foreign matter such as bacteria, viruses, nucleic acid, etc. into the sample and cross-contamination of plural samples during extraction.

### BEST MODE

Hereinafter, the present invention will be described in detail with reference to the attached drawings.

Prior to the description, according to embodiments of the present invention, the same numerals are given for the components having the same constructions, and one embodiment will be described as a representative example and other embodiments will describe only other configurations different from those in the above embodiment.

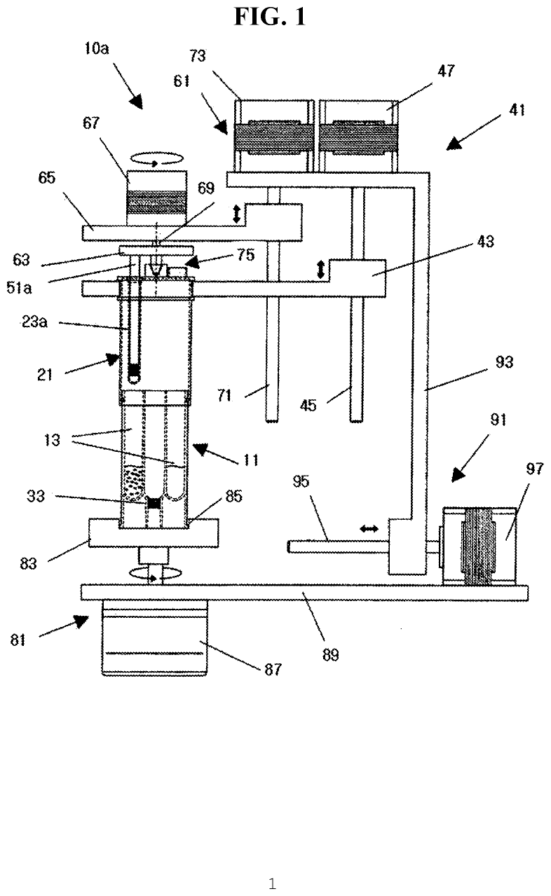

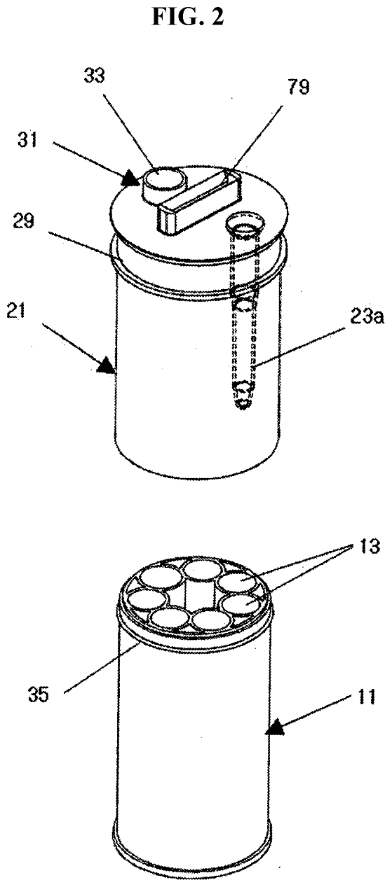

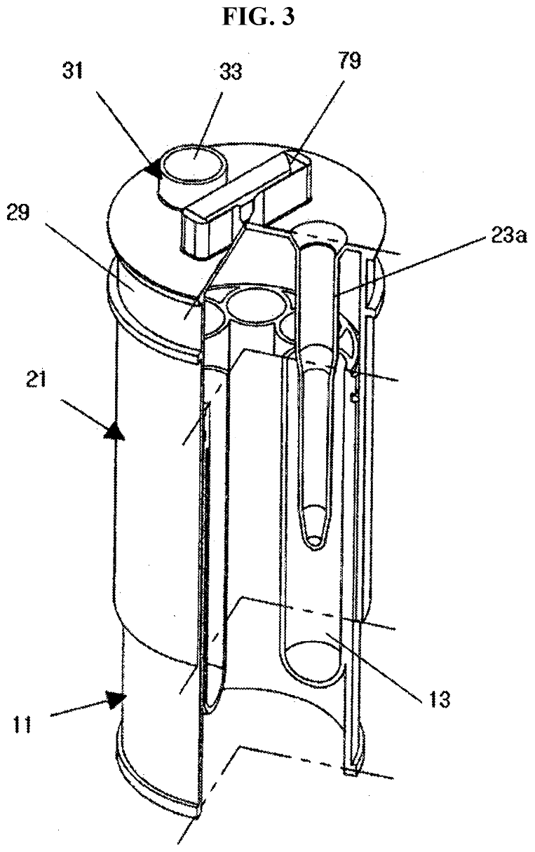

FIGS. 1 to 9 illustrate the airtight apparatus for separation and purification of an extract according to one embodiment of the present invention.

As shown in these figures, the airtight apparatus 10a for separation and purification of an extract according to one embodiment of the present invention includes a chamber cartridge 11, a lid 21, a sealing member 35, a lid driver 41, a rod 51a, a rod driver 61 and a bore moving member 75.

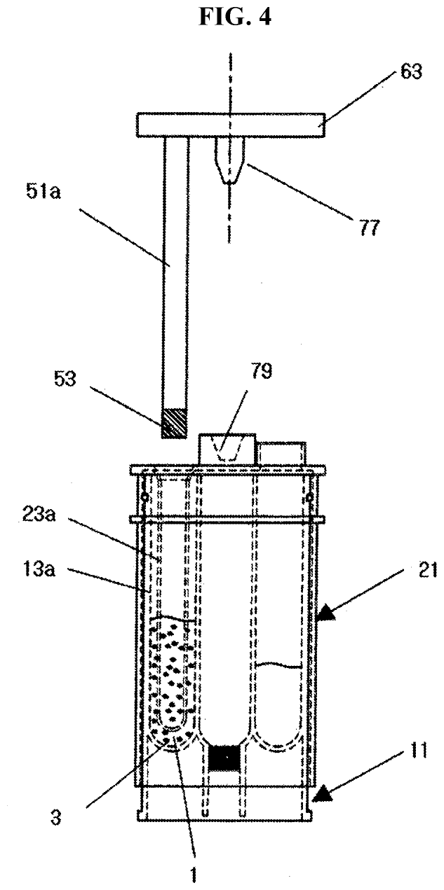

The chamber cartridge 11 may have a cylindrical shape. The chamber cartridge may be provided with a plurality of chambers 13 to receive a sample 1 (see FIG. 4) and magnetic beads 3 (see FIG. 4) in order to separate a biological material. Each of the plurality of chambers 13 may have an opening opened on top of the chamber cartridge 11, through which the sample 1 and the magnetic beads are introduced into and withdrawn from the chamber 13. The plurality of chambers 13 may be arranged at a distance along a periphery of the chamber cartridge 11. In this case, the sample 1 collectively refers to a substance made of liquid, solid or a mixture of liquid and solid in relation to a biological or chemical process.

The lid 21 may have a hollow cylindrical shape opened at one side thereof. The lid 21 may have an inner diameter substantially the same as or slightly larger than an outer diameter of the chamber cartridge 11. The lid 21 may cover the plurality of chambers 13 and be lift-possibly coupled to the chamber cartridge 11.

A bore 23a introducible into (or entering) any one of the plurality of chambers 13 may be provided on the inside of the lid 21 facing the chamber cartridge 11.

In the present embodiment, the bore 23a may have a tube shape with a hollow cross-section opened at one side thereof. The bore 23a may protrude from the inside of the lid 21 toward the chamber cartridge 11 in order to expose the opening of the bore to the top of the lid 21. The bore 23a has a relatively smaller diameter than that of the chamber 13. Therefore, the bore 23 can be inserted into the chamber 13 while forming a space between the bore and an inner peripheral side of the chamber 13 wherein the sample 1 or a buffer may flow along the space. Further, the rod 51a may be inserted into and withdrawn from the inside of the bore 23a.

Further, a bracket coupling member 29 may be provided along a periphery of the lid 21 on top of the lid 21, and the bracket coupling member 29 may be coupled to a lid bracket 43 described later.

Further, an air vent part 31 may be provided on top surface of the lid 21 to discharge air between the lid 21 and the chamber cartridge 11 to the outside. The air vent part 31 may serve to guide the air present between the lid 21 and the chamber cartridge to be smoothly discharged to the outside when the lid 21 descends to the chamber cartridge 11.

Further, the air vent part 31 may be provided with a filter member 33 on a flow path through which the air is discharged. The filter member 33 may serve to prevent foreign matter and viruses from inflowing between the chamber cartridge 11 and the lid 21. Further, the filter member 33 may be provided in the bottom center of the chamber cartridge 11.

Herein, the present embodiment illustrated the air-vent part 31 provided in the lid 21 without particular limitation thereof. That is, instead, the air-vent part 31 may be provided in the chamber cartridge 11. Further, the air-vent part 31 may be designed in a thread line manner to be opened and closed, thereby enabling recovery of the extract after completing inflow and extraction of a sample.

The sealing member 35 may be provided between the lid 21 and the chamber cartridge 11, more particularly, between an inner peripheral side of the lid 21 and an outer peripheral side of the chamber cartridge 11 to hermetically seal a space between the lid 21 and the chamber cartridge 11. In the present embodiment, an O-ring is illustrated as the sealing member 35 but the embodiment is not limited thereto. As such, since the lid 21 and the chamber cartridge 11 are hermetically sealed by the sealing member 35, extraction may be performed without being exposed to air. Therefore, it is possible to prevent infection of a subject under experiment by an infectious agent contained in the sample 1, or an infectious agent in air from inflowing in the sample 1.

The lid driver 41 may lift up the lid 21 such that the bore 23a can ascend from and descend to the chamber 13. The lid driver 41 may include a lid bracket 43 for supporting the lid 21, a lid screw shaft 45 for lifting the lid bracket 43 up and down, and a lid drive motor 47 for rotating the lid screw shaft 45.

The lid bracket 43 may have an arm shape and support the bore 23a in a suspended manner

The lid screw shaft 45 may have a bar shape with a predetermined length, and a male screw may be formed around an outer periphery of the lid screw shaft 45 to be screw-coupled to the lid bracket 43. The lid screw shaft 45 is screw-rotatably coupled to the lid bracket 43, thus lifting the lid bracket 43 up and down. The lid drive motor 47 may be directly connected to the lid screw shaft 45 by a coupling (not shown) to forwardly and reversely rotate the lid screw shaft 45. According to forward/reverse rotation of the lid screw shaft 45, the lid bracket 43 may ascend from and descend to the chamber cartridge 11, whereby the bore 23a in the lid 21 may ascend from and descend to any one of the plurality of chambers 13 in the chamber cartridge 11.

The rod 51a may have a bar shape with a predetermined length, and be inserted into and withdrawn from the bore 23a

The rod 51a is coaxial to the bore 23a and is disposed above the bore 23a, and is supported in a suspended manner to the first rod bracket 63 of the rod driver 61 described later. According to the present embodiment, a magnet 53 for generating a magnetic force in order to couple to the magnetic beads 3 may be provided at a free end of the rod 51a. Therefore, when the rod 51a is inserted into the bore 23a, the magnetic beads 3 received in the chamber 13 of the chamber cartridge 11 may be adhered to the surface of the bore 23a by the magnetic force of the rod 51a. In contrast, when the rod 51a is withdrawn from the bore 23a, the magnetic beads adhered to the surface of the bore 23a may be isolated from the surface of the bore 23a due to loss of the magnetic force of the rod 51a, thereby being released and entering the chamber 13 of the chamber cartridge 11.

The rod driver 61 may insert/withdraw the rod 51a into/from the bore 23a The rod driver 61 may include a first rod bracket 63, a second rod bracket 65, a bracket drive motor 67, a rod screw shaft 71 and a rod drive motor 73.

The first rod bracket 63 may have a disc shape and support the rod 51a in a suspended manner at a position in response to the bore 23a.

The second rod bracket 65 may have an arm shape and be arranged at a distance above the first rod bracket 63.

The bracket drive motor 67 has a body supported by the second rod bracket 65 and is coupled to a motor shaft of the bracket drive motor 67, for example, the drive shaft 69 is coupled to the first rod bracket 63. Thus, when the bracket drive motor 67 forwardly and reversely rotates, the first rod bracket 63 may rotate in one direction or the other direction and, at the same time, the rod 51a may also rotate in the one direction or the other direction.

The rod screw shaft 71 may have a bar shape with a predetermined length, and a male screw coupled to the second rod bracket 65 may be formed around an outer periphery of the rod screw shaft 71. The rod screw shaft 71 may be screw-rotatably coupled to the second rod bracket 65, thereby lifting the second rod bracket 65 up and down.

The rod drive motor 73 may be directly connected to the rod screw shaft 71 by a coupling (not shown) in order to forwardly and reversely rotate the rod screw shaft 71. According to forward and reverse rotation of the rod screw shaft 71, the second rod bracket 65 and the first rod bracket 63 may ascend from and descend to the chamber cartridge 11 while the rod 51a disposed above the bore 23a may move into or out of the bore 23a.

The bore moving member 75 may move the bore 23a to any one of the plurality of chambers 13. The bore moving member 75 may include a key 77 protruding from the first rod bracket 63 toward the lid 21 and a key coupling member 79 disposed on the lid 21, wherein the key 77 is engaged with and released from the key coupling member.

The key 77 is provided on a coaxial line with the drive shaft 69 of the bracket drive motor.

Meanwhile, the key 77 may be engaged with and released from the key coupling member 79 by the lifting operation of the rod driver 61. Thus, when the key 77 is engaged with the key coupling member 79 and the bracket drive motor 67 rotates in one direction, the lid 21 and the first rod bracket 63 may rotate about the drive shaft 69 of the bracket drive motor 67 and, at the same time, the bore 23a and the rod 51a may also rotate about the drive shaft 69, whereby the bore 23a and the rod 51a may move toward a top of any one of the plurality of chambers 13. Further, the airtight apparatus 10a for separation and purification of an extract according to one embodiment of the present invention may further include an orbital shaker 81 in which the chamber cartridge 11 is seated and which performs horizontal trajectory movement of the chamber cartridge 11.

The orbital shaker 81 may include a shaking platform 83 and a shaking motor 87.

The shaking platform 83 may have a flat plate shape and a seating groove 85, in which the chamber cartridge 11 is seated, given in a depression form. The shaking motor 87 is supported on a base 89 and directly connected to the shaking platform 83 by a coupling (not shown), thereby enabling horizontal trajectory movement of the shaking platform 83.

Accordingly, the sample 1 and the magnetic beads 3 received in the chamber 13 of the chamber cartridge 11, which is seated in the shaking platform 83, may flow along a wall face of the chamber 13 by driving the shaking motor 87, whereby the sample 1 and the magnetic beads 3 can be flow-mixed at a high speed.

Further, the airtight apparatus 10a for separation and purification of an extract according to one embodiment of the present invention may further include a reciprocation member 91 to reciprocate the lid driver 41 and the rod driver 61 in a transverse direction to the lifting direction of the bore 23a.

The reciprocation member 91 may include a main support bracket 93, a main screw shaft 95 and a main drive motor 97.

The main support bracket 93 supports the lid driver 41 and the rod driver 61. More particularly, the rod screw shaft 71 and the lid screw shaft 45 may pass through and be screw-coupled to one end of the main support bracket 93 while supporting the lid drive motor 47 and the rod drive motor 73. Further, a main screw shaft 95 may pass through and be screw-coupled to the other end of the main support bracket 93.

The main screw shaft 95 may have a bar shape with a predetermined length. Further, a male screw may be formed around an outer periphery of the main screw shaft 95 to be screw-coupled to the main support bracket 93. The main screw shaft 95 is screw-rotatably coupled to the main support bracket 93, whereby the main support bracket 93 may be reciprocated in a transverse direction to the lifting direction of the bore 23a, for example, the lid 21 and the rod 51a may be reciprocated in a transverse direction to the lifting direction of the bore 23a.

The main drive motor 97 is directly connected to the main screw shaft 95 by a coupling (not shown), thereby forwardly and reversely rotating the main screw shaft 95. The main drive motor 97 may be supported on a base 89, and the shaking motor 87 of the orbital shaker 81 may further be supported on the base 89. Thus, when the bore 23a and the rod 51a are disposed on top of the chamber cartridge 11 while being spaced from the chamber cartridge 11, the bore 23a and the rod 51a may be reciprocated in a transverse direction to the lifting direction of the bore 23a by forwardly and reversely rotating the main screw shaft 95.

Using the airtight apparatus 10a for separation and purification of an extract with the configurations described above according to one embodiment of the present invention, a process of separating and purifying diverse biological materials such as nucleic acid or antibodies as an extract from a sample 1 will be briefly described later.

First, while the chamber cartridge 11 and the lid 21 are separated, a lysed buffer able to dissolve the biological material such as nucleic acid or antibodies to be separated, as well as magnetic beads 3, may be received in a first chamber 13a in the chamber cartridge 11. Further, an elution buffer may be received in a second chamber 13b adjacent to the first chamber. Further, after opening the air-vent part 31 which can be opened and closed, the extracted sample is introduced into the first chamber 13a through the air-vent part 31.

Next, the chamber cartridge 11 is seated on the shaking platform 83 of the orbital shaker 81. At the same time, the lid 21 is disposed above the chamber cartridge 11 such that the bore 23a is positioned on top of the first chamber 13a. Herein, after placing the chamber cartridge 11 on the shaking platform 83, the sample 1 and the magnetic beads 3 are received in the first chamber 13a while the second chamber 13b may include the elution buffer.

Then, the lid drive motor 47 forwardly and reversely rotates such that the lid 21 ascends and descends along an outer periphery of the chamber cartridge 11. By the forward and reverse rotation of the lid drive motor 47, the lid screw shaft 45 may be forwardly and reversely rotated, whereby the lid 21 supported by the lid bracket 43 may ascend and descend in the screw shaft direction.

Accordingly, the bore 23a provided inside the lid 21 may be under lifting movement in the first chamber 13a, while the magnetic beads 3 and the sample 1 received in the first chamber 13a may collide with each other by the lifting movement of the bore 23a.

Simultaneously, driving the shaking motor 87 of the orbital shaker 81 may perform horizontal trajectory movement of the chamber cartridge 11, thereby enabling a collision between the magnetic beads 3 and the sample received in the first chamber 13a.

As such, due to horizontal trajectory movement of the orbital shaker 81 along with the lifting movement of the bore 23a, the magnetic beads 3 and the sample 1 received in the first chamber 13a may more vigorously collide with each other, thereby adsorbing the biological material such as nucleic acid or antibodies contained in the sample 1 onto the magnetic beads 3.

On the other hand, although the lid 21 and a top surface of the chamber cartridge 11 are kept airtight by the sealing member 35, air present between the lid 21 and the top surface of the chamber cartridge 11 moves through the air-vent part 31 during lifting of the lid 21, whereby the lid 21 may smoothly ascend from and descend to the chamber cartridge 11.

Therefore, when the biological material such as nucleic acid or antibodies contained in the sample 1 is adsorbed onto the magnetic beads 3 due to horizontal trajectory movement of the orbital shaker 81 as well as the lifting movement of the bore 23a, as shown in FIG. 4, the second rod bracket 65 may descend toward the lid 21 by rotating the rod drive motor 73 in one direction while receiving the bore in the first chamber 13a.

Upon lifting down the second rod bracket 65, the rod 51a supported by the first rod bracket 63 may descend and be received in the bore 23a. Herein, the key 77 provided in the first rod bracket 63 is engaged with the key coupling member 79 on the lid 21, whereby the first rod bracket 63 and the lid 21 may be maintained in an interconnected state.

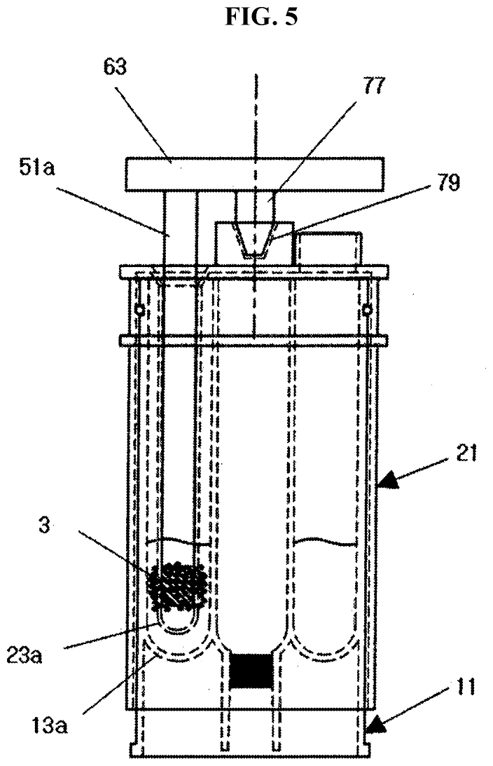

Meanwhile, since the rod 51a is received in the bore 23a, as shown in FIG. 5, the magnetic beads 3 including the biological material adsorbed thereon may be adhered to an outer surface of the bore 23a by magnetic force of the magnet 53 provided at a free end of the rod 51a.

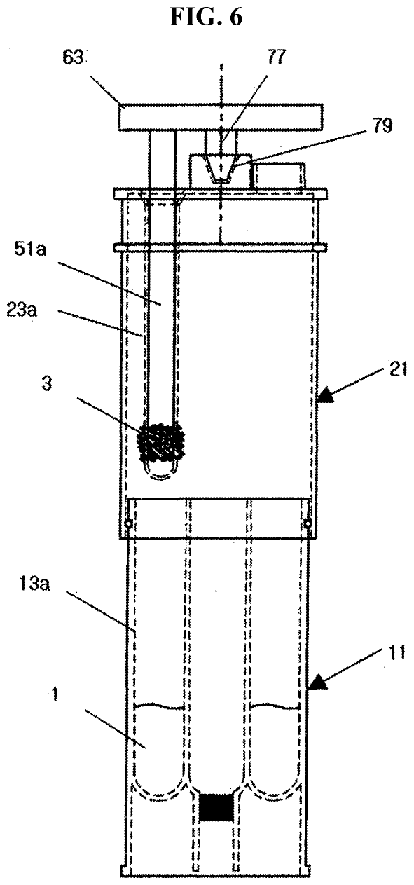

Further, after completing adhesion of the magnetic beads 3 to the outer surface of the bore 23a, as shown in FIG. 6, the lid 21 may ascend by rotating the lid drive motor 47 in the other direction so as to withdraw the bore 23a from the first chamber 13a and, at the same time, the second rod bracket 65 may ascend by rotating the rod drive motor 73 in the other direction so as to withdraw the rod 51a from the first chamber 13a. In this case, the lid drive motor 47 and the rod drive motor 73 may be synchronized to lift up the bore 23a and the rod 51a simultaneously.

Since the bore 23a and the rod 51a are withdrawn from the first chamber 13a, separation of the biological material from the first chamber 13a may be completed.

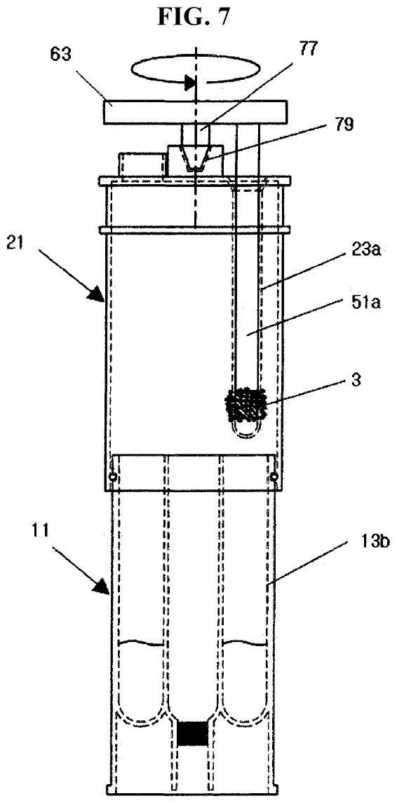

Subsequently, while the key 77 provided in the first rod bracket 63 is engaged with the key coupling member 79, the bracket drive motor may rotate in one direction so as to move the bore 23a including the magnetic beads 3 adhered thereto as well as the rod 51a above the second chamber 13b, as shown in FIG. 7. Herein, the elution buffer may be received in the second chamber 13b.

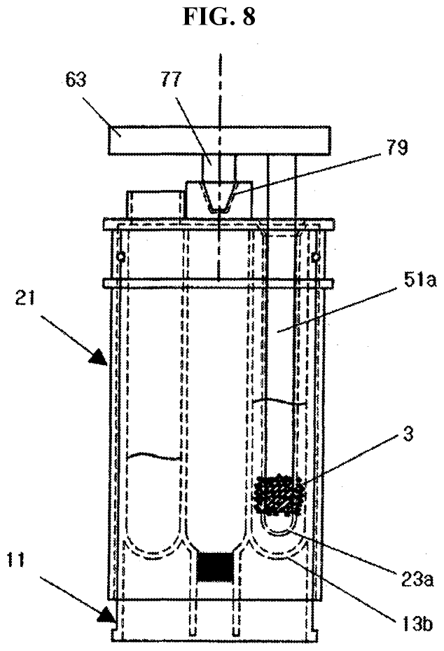

Then, the lid 21 may descend by rotating the lid drive motor 47 in one direction so as to insert the bore 23a into the second chamber 13b and, at the same time, the second rod bracket 65 may descend by rotating the rod drive motor 73 in one direction so as to insert the rod 51a into the second chamber 13b, as shown in FIG. 8. In this case, the lid drive motor 47 and the rod drive motor 73 may be synchronized to lift down the bore 23a and the rod 51a simultaneously.

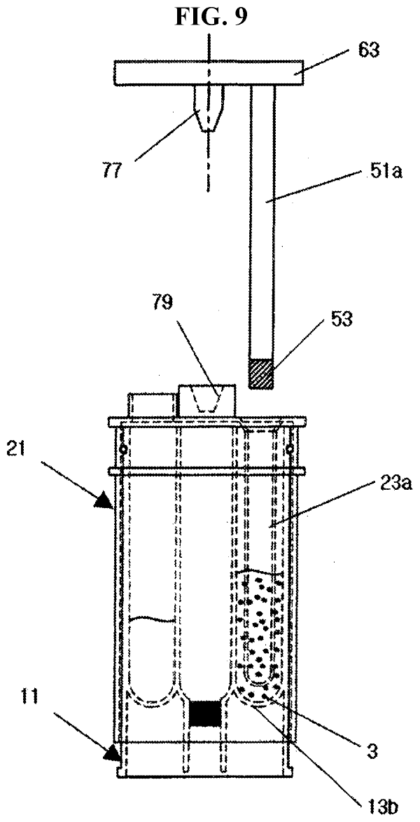

On the other hand, rotating the rod drive motor 73 in the other direction may lift up the second rod bracket 65 to be apart from the lid 21. Upon lifting up the second rod bracket 65, the rod 51a supported by the first rod bracket 63 may ascend and be withdrawn from the bore 23a, as shown in FIG. 9. Due to withdrawal of the rod 51a from the bore 23a, the magnetic force acting on the magnetic beads 3 adhered to the outer surface of the bore 23a is lost, whereby the magnetic beads 3 may be released from the surface of the bore 23a while being dispersed into the elution buffer in the second chamber 13b.

Further, pure magnetic beads 3 and the biological materials may be separated from each other by the elution buffer in the second chamber 13b.

After completing the separation of the biological materials from the pure magnetic beads 3 in the second chamber 13b, the second rod bracket 65 descends toward the lid 21 by again rotating the rod drive motor 73 in one direction, although not shown in the drawings.

Upon lifting down the second rod bracket 65, the rod 51a supported by the first rod bracket 63 may descend and be received in the bore 23a. In this case, the key 77 provided in the first rod bracket 63 may be engaged with the key coupling member 79 of the lid 21, whereby the first rod bracket 63 and the lid 21 may be maintained in an interconnected state.

Meanwhile, since the rod 51a is received in the bore 23a, pure magnetic beads 3 free of the biological material may be adhered to the outer surface of the bore 23a by the magnetic force of the magnet 53 provided at a free end of the rod 51a.

Further, upon completion of the adhesion of the pure magnetic beads 3 to the outer surface of the bore 23a, the lid 21 may ascend by rotating the lid drive motor 47 in the other direction so as to withdraw the bore 23a from the second chamber 13b and, at the same time, the second rod bracket 65 may ascend by rotating the rod drive motor 73 in the other direction so as to withdraw the rod 51a from the second chamber 13b. In this case, the lid drive motor 47 and the rod drive motor 73 may be synchronized so as to lift up the bore 23a and the rod 51a simultaneously.

Since the bore 23a and the rod 51a are withdrawn from the second chamber 13b, the magnetic beads 3 may be recovered from the second chamber 13b, while the biological material such as pure nucleic acid or antibodies only is left in the second chamber 13b.

As such, a process for extraction and purification of biological materials such as nucleic acid or antibodies is completed.

In this way, a series of processes for separation and purification of an extract from the sample 1 may be implemented in a closed state (or airtight state) without exposing the sample 1 to air, and may prevent infection of a subject under experiment by an infectious agent contained in the sample 1 or introduction of any external infectious agent present in air into the sample 1.

In this regard, the present embodiment described purification of biological materials using an elution buffer after separation of the same from the sample 1. However, prior to purification of the biological material using the elution buffer, the foreign matter adsorbed onto the magnetic beads 3 along with the biological material such as nucleic acid or antibodies may be washed beforehand using a washing buffer. Thereafter, the washed product may be subjected to the purification using the elution buffer as described above.

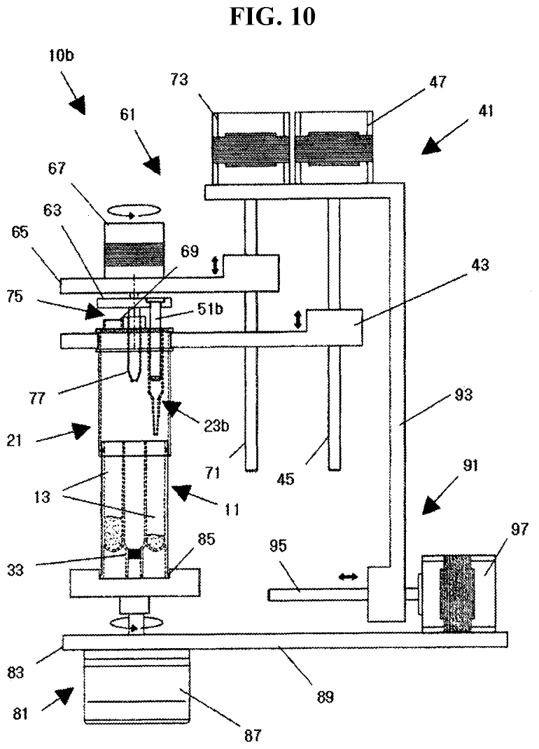

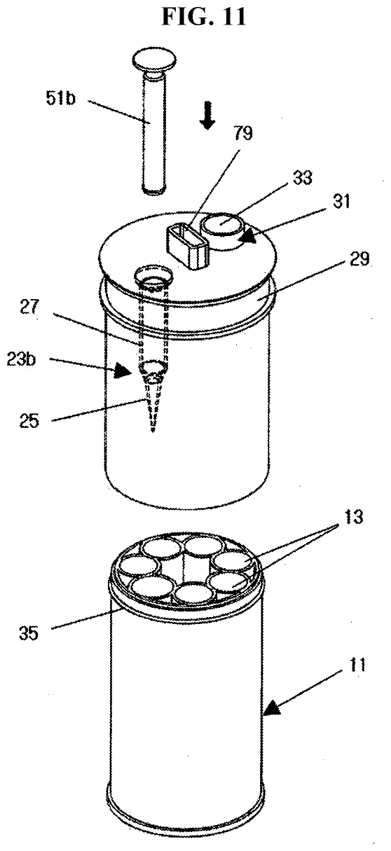

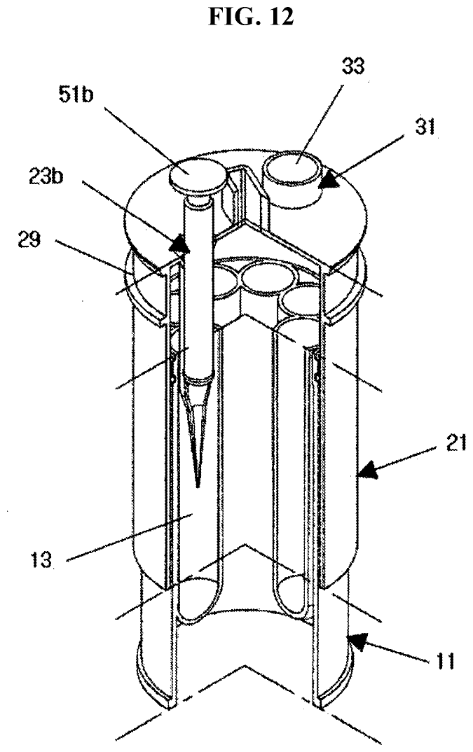

Meanwhile, FIGS. 10 to 18 illustrate the airtight apparatus for separation and purification of an extract according to another embodiment of the present invention.

As shown in these figures, the airtight apparatus for separation and purification of an extract according to this embodiment is different from the previous embodiment, and includes a bore 23b in a cylindrical syringe shape. That is, the bore 23b may include a needle part 25 in a hollow conical shape having a sharp distal end and a cylinder part 27 in a hollow cylindrical column shape in communication with the needle part 25.

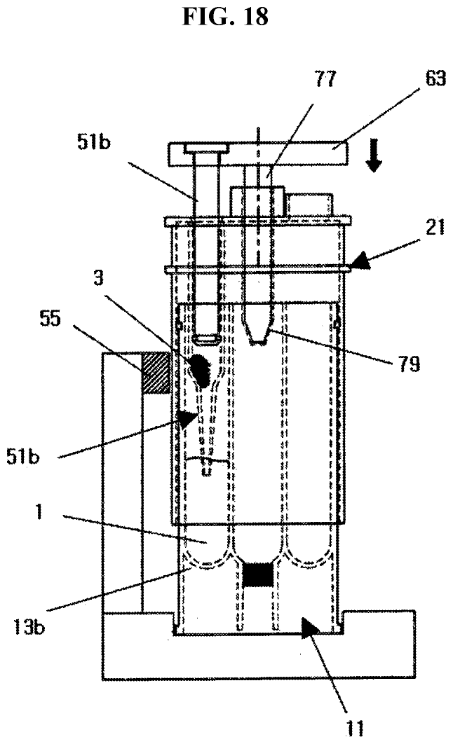

Further, unlike the previous embodiment, the present embodiment describes the apparatus including a rod 51b in a piston shape in sliding contact with an inner periphery of the cylinder part 27 of the bore 23b without a magnet. The rod 51b may serve to introduce/discharge the biological materials 5 (see FIG. 18) as well as magnetic beads 3 into/from the bore 23b. The rod 51b may ascend and descend while being in an airtight state inside the bore 23b by fitting an O-ring (not shown) or a rubber ring (not shown) in a bottom of the rod 51b, thereby introducing/discharging the sample 1 or the magnetic beads 3 into/from the bore 23b.

Further, the airtight apparatus 10b for separation and purification of an extract according to another embodiment of the present invention (hereinafter, “the apparatus 10b of the present embodiment”) may further include a magnet 55 to attach/detach the magnetic beads 3 received in the bore 23b to/from a surface of the bore 23b.

The magnet 55 may be provided on an outer side of a chamber 13 which is used for separation. According to the present embodiment, the magnet 55 is disposed on the outer side of the chamber 13 by a support 57 vertical to a shaking platform 83.

Using the airtight apparatus 10b for separation and purification of an extract with the configurations described above according to the present embodiment, a process of separating and purifying diverse biological materials such as nucleic acid or antibodies contained in cells of a sample 1 will be briefly described later.

First, while a chamber cartridge 11 and a lid 21 are separated, a buffer able to dissolve the biological material such as nucleic acid or antibodies to be separated, as well as magnetic beads 3, may be received in a first chamber 13a in the chamber cartridge 11. A second chamber 13b adjacent to the first chamber may be empty. Further, after opening an air-vent part 31 which can be opened and closed, the sample to be extracted is introduced into the first chamber 13a. Herein, the second chamber 13b may further include additional buffer therein. Next, the chamber cartridge 11 is seated on a shaking platform 83 of an orbital shaker 81. At the same time, the lid 21 is disposed above the chamber cartridge 11 such that the bore 23b is positioned on top of the first chamber 13a.

Then, the lid drive motor 47 is forwardly and reversely rotated such that the lid 21 ascends and descends along an outer periphery of the chamber cartridge 11. At the same time, the bore 23b inside the lid 21 may enter the sample 1 in the first chamber 13a, while the sample 1 and the magnetic beads 3 received in the first chamber 13a may collide with each other since the sample 1 repeatedly inflows/outflows into/from the bore 23b by the lifting movement of the rod 51b.

Simultaneously, driving the shaking motor 87 of the orbital shaker 81 may perform horizontal trajectory movement of the chamber cartridge 11, thereby enabling collision between the magnetic beads 3 and the sample received in the first chamber 13a.

As such, due to horizontal trajectory movement of the orbital shaker 81 along with the lifting movement of the bore 23a, the magnetic beads 3 and the sample received in the first chamber 13a may collide with each other, which in turn causes collision of cells in the sample 1 to separate a biological material such as nucleic acid or antibodies contained in the cells, thereby binding (or adsorbing) the biological material such as nucleic acid or antibodies contained in the sample 1 onto the magnetic beads 3.

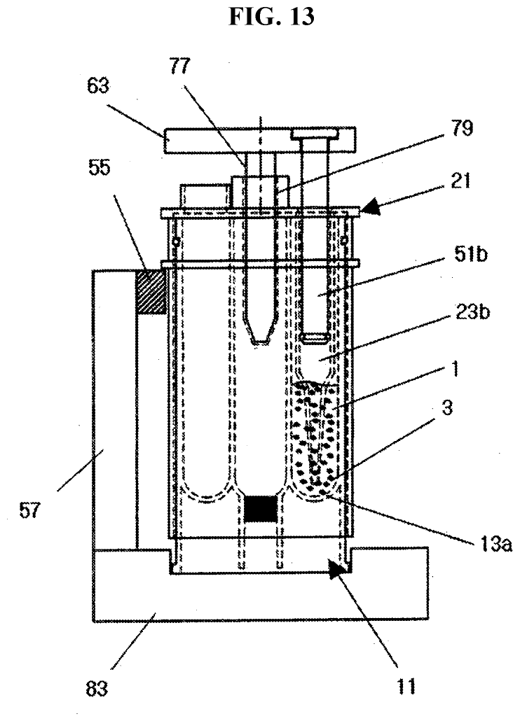

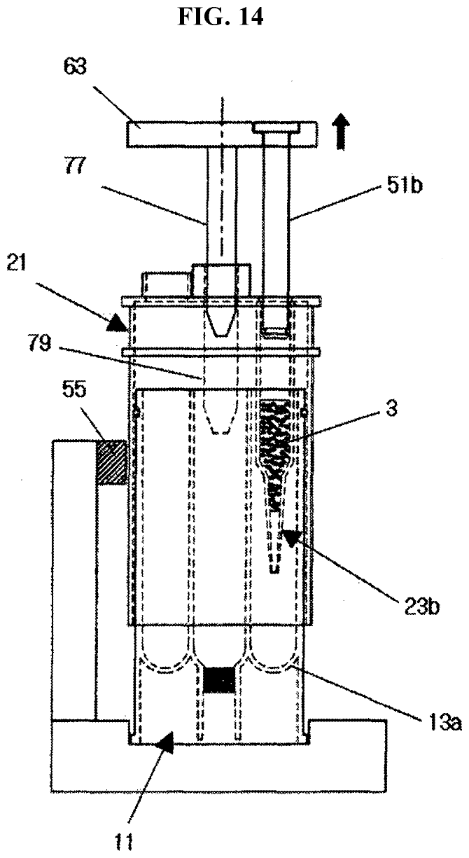

Following this, as shown in FIG. 13, when the rod drive motor 73 rotates in the other direction while receiving the bore 23b and the rod 51 in the first chamber 13a, the second rod bracket 65 may ascend and be apart from the lid 21. Upon lifting up the second rod bracket 65, the rod 51b supported by the first rod bracket 63 may ascend and, as shown in FIG. 14, the sample 1 free of the biological materials and the magnetic beads including the biological material adsorbed thereon may be introduced into the bore 23b through the needle part 25 of the bore 23b. Herein, the key 77 provided in the first rod bracket 63 is engaged with the key coupling member 79 of the lid 21 so as to maintain the first rod bracket 63 and the lid 21 in an interconnected state.

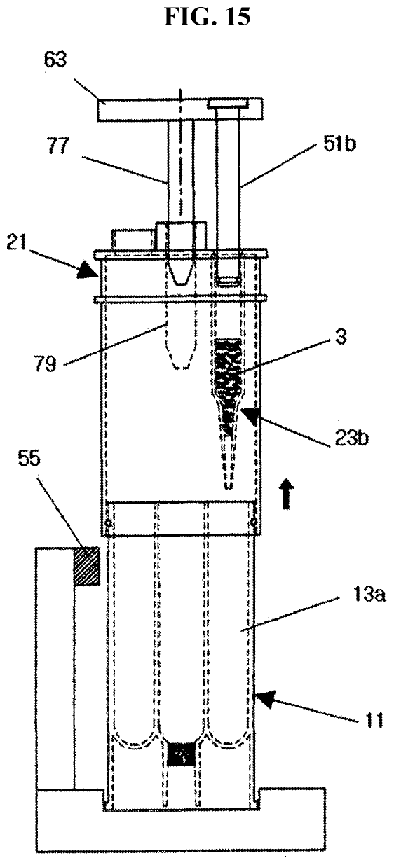

Further, when introduction of the magnetic beads 3 including the biological material adsorbed thereon as well as the sample 1 free of the biological material into the bore 23b is completed, as shown in FIG. 15, the lid 21 may ascend by rotating the lid drive motor 47 in the other direction so as to withdraw the bore 23b from the first chamber 13a and, at the same time, the second rod bracket 65 may ascend by rotating the rod drive motor 73 in the other direction so as to withdraw the rod 51b from the first chamber 13a. In this case, the lid drive motor 47 and the rod drive motor 73 may be synchronized to lift up the bore 23b and the rod 51b simultaneously.

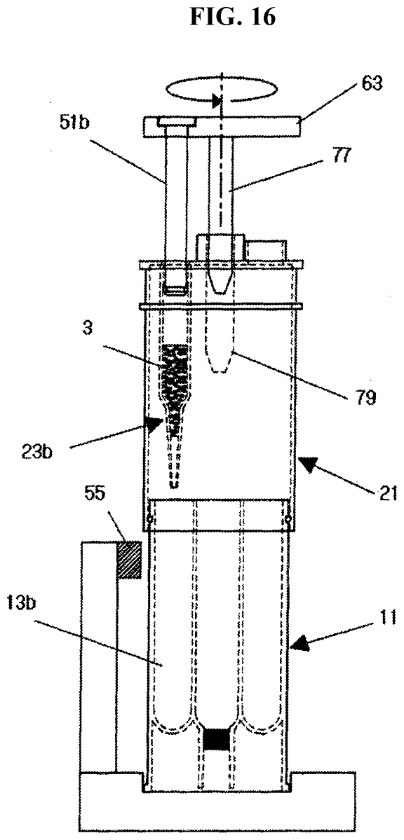

Then, while the key 77 is engaged with the key coupling member 79 of the lid 21, the bracket drive motor 67 may rotate in one direction so as to move the bore 23b as well as the rod 51b above the second chamber 13b, as shown in FIG. 16.

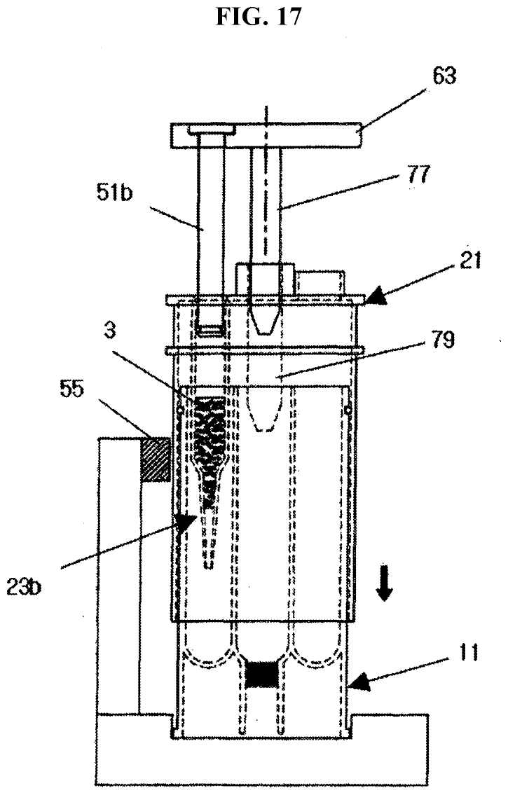

Following this, as shown in FIG. 17, the lid 21 may descend by rotating the lid drive motor 47 in one direction so as to insert the bore 23b into the second chamber 13b and, at the same time, the second rod bracket 65 may descend by rotating the rod drive motor 73 in one direction so as to insert the rod 51b into the second chamber 13b. In this case, the lid drive motor 47 and the rod drive motor 73 may be synchronized to lift down the bore 23b and the rod 51b simultaneously. Further, the second rod bracket 65 may descend toward the lid 21 by rotating the rod drive motor 73 in one direction. Upon lifting down the second rod bracket 65, as shown in FIG. 18, the rod 51b supported by the first rod bracket 63 may descend whereby the sample 1 free of the biological material, which is introduced into the bore 23b, may move toward the second chamber 13b. Alternatively, when the rod 51b descends, the magnetic beads 3 including the biological material adsorbed thereon may remain inside the bore 23b by magnetic force of the magnet 55, while discharging the sample 1 free of the biological material only from the bore 23b and collecting the same in the second chamber 13b.

As such, when the sample 1 free of the biological material and the magnetic beads 3 including the biological material adsorbed thereon are completely separated, the lid 21 may ascend by rotating the lid drive motor 47 in the other direction so as to withdraw the bore 23b from the second chamber 13b and, at the same time, the second rod bracket 65 may ascend by rotating the rod drive motor 73 in the other direction so as to withdraw the rod 51b from the second chamber 13b, although not shown in the drawings. In this case, the magnetic beads 3 including the biological material adsorbed thereon, which are received in the bore 23b, may remain inside the bore 23b without being discharged toward the second chamber 13b by a magnetic force generated between the magnetic beads 3 even if the magnetic force of the magnet 55 is lost. Herein, the lid drive motor 47 and the rod drive motor 73 may be synchronized to lift up the bore 23b and the rod 51b simultaneously.

Since the bore 23b and the rod 51b are withdrawn from the second chamber 13b, the magnetic beads 3 including the biological material adsorbed thereon may be recovered in the bore 23b while the sample 1 free of the biological material only is left in the second chamber 13b. If the magnetic beads 3 remain inside the bore 23b, the bore 23b, in which the magnetic beads are left, as well as the rod 51b may move above a third chamber (not shown) by rotating the bracket drive motor 67 in one direction while coupling the key 77 provided in the first rod bracket 63 to the key coupling member 70 of the lid 21. The third chamber may include an elution buffer.

Subsequently, the lid 21 may descend by rotating the lid drive motor 47 in one direction so as to insert the bore 23a into the elution buffer in the third chamber. Thereafter, the rod drive motor 73 forwardly and reversely rotates to lift the rod 51b up and down along the bore 23b, whereby the elution buffer in the third chamber and the magnetic beads in the bore 23b are mixed together. By repeatedly lifting the rod 51b up and down, the biological material may be isolated from the magnetic beads 3 and dispersed into the elution buffer. Although not shown in the drawings, when the pure magnetic beads 3 and the biological material are completely separated in the third chamber, the rod 51b may ascend by again rotating the rod drive motor 73 in the other direction to move the magnetic beads and the elution buffer into the bore 23b, and then, the lid 21 may ascend by rotating the lid drive motor 47 in the other direction to withdraw the bore 23b from the third chamber. At the same time, the magnetic beads 3 are gathered at one side of the bore 23b using the magnet and then the elution buffer only is gathered in the third chamber by lifting down the rod 51b. Further, according to the same procedures as described above, the bore 23b moves into the second chamber 13b, thereby completing extraction and purification of the biological material such as nucleic acid or antibodies.

Accordingly, the extraction and separation of the biological material such as nucleic acid or antibodies from the sample 1 may be completed.

As such, a process of separating an extract of biological material from the sample 1 may be implemented in an airtight state without exposing the sample 1 to air, and may prevent infection of a subject under experiment by an infectious agent contained in the sample 1 or introduction of foreign matter possibly present in the air such as bacteria, viruses or nucleic acid into the sample 1.

In this regard, the present embodiment described the process of separating diverse biological materials such as nucleic acid or antibodies contained in cells of the sample 1. However, the airtight apparatus 10b for separation and purification of an extract according to another embodiment of the present invention may also be used in a process for purification of the biological material such as nucleic acid or antibodies isolated from cells wherein this process may also be implemented in the same manner as described above.

Further, the present invention may be applied to an integral apparatus for extraction and diagnosis, wherein: a fluorescent material for polymerase chain reaction (PCR) enabling nucleic acid amplification is introduced beforehand to any one of the chambers; an aluminum block able to increase and decrease a temperature and a detector able to detect the fluorescent material are disposed on the bottom of the chamber; and the extracted nucleic acid is partially delivered to the above chamber to perform PCR, followed by detecting nucleic acid amplification in real time.

What is claimed is:

1. An airtight apparatus for separation and purification of an extract, comprising:
a chamber cartridge having a plurality of chambers for receiving a sample and magnetic beads for separation of a biological material;
a lid having a bore through which the sample can be introduced into any one of the plurality of chambers, covering the plurality of chambers, and being lift-possibly coupled to the chamber cartridge;
a sealing member provided between the lid and the chamber cartridge to keep a space between the lid and a housing airtight;
a lid driver for lifting the lid up and down such that the bore ascends and descends in the chamber;
a rod inserted into and withdrawn from the bore to attach/detach the biological material adsorbed onto the magnetic beads present in the chamber to/from the surface of the bore or to introduce/discharge the sample free of the biological material and the magnetic beds into/from the bores;
a rod driver for inserting and withdrawing the rod into/from the bore; and
a bore moving member for moving the bore to any one of the plurality of chambers.

2. The apparatus according to claim 1, further comprising a magnet to attach/detach the magnetic beads received in the bore to/from the surface of the bore.

3. The apparatus according to claim 1, wherein the lid driver includes:
a lid bracket to support the lid;
a lid screw shaft screw-rotatably coupled to the lid bracket to lift the same up and down; and
a lid drive motor to rotate the lid screw shaft.

4. The apparatus according to claim 1, wherein the rod driver includes:
a first rod bracket for supporting the rod;
a second rod bracket disposed apart from the first rod bracket at a distance;
a bracket drive motor supported by the second rod bracket, having a drive shaft coupled to the first rod bracket, and rotating the first rod bracket;
a rod screw shaft screw-rotatably coupled to the second rod bracket to lift the same up and down; and
a rod drive motor for rotating the rod screw shaft.

5. The apparatus according to claim 4, wherein the bore moving member includes:
a key protruding from the first rod bracket toward the lid; and
a key coupling member disposed on the lid by which the key is engaged and released.

6. The apparatus according to claim 1, further comprising: a reciprocation member to reciprocate the lid driver and the rod driver in a transverse direction to the lifting direction of the bore.

7. The apparatus according to claim 6, wherein the reciprocation member includes:
a main support bracket to support the lid driver and the rod driver;
a main screw shaft screw-rotatably coupled to the main support bracket to reciprocate the main support bracket in a transverse direction to the lifting direction of the bore; and
a main drive motor for rotating the main screw shaft.

8. The apparatus according to claim 1, further comprising: an orbital shaker, on which the chamber cartridge is seated, to enable horizontal trajectory movement of the chamber cartridge.

9. The apparatus according to claim 8, wherein the orbital shaker includes:
a shaking platform on which the chamber cartridge is disposed, and
a shaking motor to conduct the horizontal trajectory movement of the shaking platform.

10. The apparatus according to claim 1, further comprising: an air-vent part provided in the lid or the chamber cartridge to discharge the air between the lid and the chamber cartridge to an outside and introduce fresh air from the outside.

11. The apparatus according to claim 10, wherein the air-vent part further includes a filter member to block introduction of foreign matter such as bacteria, viruses, nucleic acid, etc. and/or outflow of an aerosol including nucleic acid or relevant samples therein.

12. The apparatus according to claim 1, wherein the bore has a tube shape opened at one side thereof.

13. The apparatus according to claim 1, wherein the bore has a cylindrical syringe shape opened at opposite sides.

14. The apparatus according to claim 1, further comprising: a magnet provided below the rod to attach/detach the magnetic beads to/from the surface of the bore.

15. The apparatus according to claim 1, wherein an O-ring or a rubber ring is adhered to a lower portion of the rod so that the rod ascends and descends while keeping the inside of the bore airtight, thereby introducing/discharging the sample or the magnetic beads to/from the bore.
